(19) United States
(12) Patent Application Publication
(10) Pub. No.: US 2022/0054067 A1
(43) Pub. Date: Feb. 24, 2022

(54) MINIATURIZED INDUCTION COIL-BASED NEURAL MAGNETOMETER

(71) Applicant: TRITON SYSTEMS, INC., Chelmsford, MA (US)

(72) Inventors: Adhvait SHAH, Chelmsford, MA (US); Jan BERKOW, Chelmsford, MA (US); Asimina KIOURTI, Columbus, OH (US); Keren ZHU, Columbus, OH (US)

(21) Appl. No.: 17/407,862

(22) Filed: Aug. 20, 2021

Related U.S. Application Data
(60) Provisional application No. 63/068,741, filed on Aug. 21, 2020.

(51) Int. Cl. A61B 5/30; A61B 5/248; A61B 5/00

(52) U.S. Cl. CPC A61B 5/30; A61B 5/7203; A61B 5/248

(57) ABSTRACT

An electromagnetic bio-signal detector to monitor very weak evoked action potentials associated with neurotransmissions is described. The small induction-coil array detector and integrated circuit design enables the device to have a small and possibly portable form factor while minimizing cost. Advanced signal processing methods enables the device to detect very weak electromagnetic signals without the need for shielding to reduce electromagnetic background emissions. The combination of cost, size, and sensitivity affords the electromagnetic bio-signal detector broad utility both inside and outside hospital settings and for numerous diagnostic and treatment feedback applications.

### CROSS-REFERENCE TO RELATED APPLICATIONS

This application claims priority to U.S. Provisional Application Ser. No. 63/068,741 filed on Aug. 21, 2020 titled Miniaturized Induction Coil-Based Neural Magnetometer, the contents of which are incorporated herein by reference in its entirety.

### GOVERNMENT INTEREST

This invention was made with Government support from the Department of the Army under Contract No. W81WH18C0097. The Government has certain rights in this invention.

### BACKGROUND

Nervous system transmissions (neurotransmitters) are used to control many functions in the body. Specifically, neurotransmitters are used to control the brain, heart, and other organ functions, the musculature, as well as affect psychological functions and communicate pain. The ability to capture, detect, and/or measure a neurotransmission can provide valuable information related to whether an organ or muscle is appropriately activated, the presence of a pain response, and the health of the neurotransmission network. For example, the lack of detected response from a finger pin prick may indicate the presence of lesion in the path of the neural transmission or poor nerve cell health due to inadequate perfusion.

A neurotransmission consists of weak electrical impulses produced by a cascade of cellular electrochemical impulses (also called action potentials). An electrical impulse is produced when a nerve cell membrane undergoes a shift in electric charge distribution, resulting in less negative charge inside the cell compared to the outside. The neurotransmission-related electrical impulses are typically detected using electrodes that are either inserted into the tissue near the nerve fibers or in the form of a patch attached to the skin. The use of electrodes to detect an electrical impulse has known drawbacks. For example, implanted electrodes can cause extreme tissue irritation, infection, and wound breakdown.

Additionally, given that tissue and bone are poor electrical conductors, the transmission of these weak electrical impulses are diffused in the tissue of the body. This limits the value of employing electrode-based diagnostics of this bio-signal detected in this manner to the pattern recognition based on pulse frequency and strength. Diffusion of these neurotransmissions limits the ability to locate neural dysfunctions or interrupted transmission path locations resultant of poor perfusion or nerve cell damage.

A neurotransmission can be artificially created to replace an inappropriate neurotransmission such as the use of an artificial cardiac pacemaker. It can also be used to stimulate a neural pathway to diagnose its health such as in the instance of a nerve stimulation device. When nerve cell is artificially activated, an external electrical impulse is used to cause the nerve cell membrane potential to rapidly rise and fall and is referred to as depolarization of the cell. An external electrical impulse can also be designed to block neurotransmission. This can be performed by causing the cell membrane to shift its electrical charge distribution to become more negative. When the nerve cell is maintained in this negative hyperpolarization condition, it is referred to as a refractory state and the cascading neurotransmission effect to adjacent nerve cells is halted.

An electrode-based approach has been used to block a pain response in this manner. However, a pain block has been shown to be more effective if the stimulation signal parameters are adjusted over time to accommodate physiologic changes to maintain an optimal block. Currently only electrode-based measures and detectors have been used to assess the effectiveness of a pain block to enable the device to be designed in a closed-loop or auto-adjusted manner. An electromagnetic bio-signal detector could be constructed such that it is non-invasive and provides periodic feedback to a neurotransmission device for optimal nerve block or pain reduction functionality. Adjustment to stimulation frequency, amplitude, pulse width, duty-cycle, and proximity to the nerve (or nerve bundle) can influence the effectiveness in the reduction of pain. Additionally, clinicians routinely record the evoked action potential amplitude at which the patient feels comfortable or duration that they can tolerate for a 1-minute duration. This is often used as a therapeutic dosage threshold and maximum comfort level measure. A detector that is able to be used to capture amplitudinal changes that may be the result of physical movement, neuroplasticy related changes, and location in respect to the nerve or other possible changes could be used to adjust the artificial neurotransmission to ensure that it is effective for pain therapy applications for an extended duration. Saluda Medical, for example is one of the first companies that is employing an implantable electrode-based detector as part of a closed-loop pain management device for spinal cord stimulation applications.

The pulsed electrical current associated with these neurotransmissions produce weak pulsed magnetic fields. Given that these magnetic fields are on the order of less than 10⁶ Gauss (10⁶ times lower than the earth's magnetic field), the signal must be captured in a shielded room to reduce competing ambient noise and/or advanced signal processing techniques must be employed to achieve the detection sensitivity to capture this signal. The utility afforded by a device that can detect these weak electromagnetic neurotransmissions can include: the ability to discriminate when these signals are inappropriate, indicating a dysfunction that could be resultant of a lesion, poor perfusion, or a form of neuropathy; the ability to detect a pain response and possible location of its source; the ability to provide feedback to determine optimal adjustment for an artificially produced neurotransmission to create a nerve block; the ability to capture different frequencies of brain waves patterns to correlate with brain injury symptoms or determine the presence or severity of a prior concussive or blast related event; the ability to detect a lesion or inappropriate body positioning during casualty immobilization or orthopedic surgery resulting in inadequate nerve cell perfusion; and the ability to detect a poor cardiac pace-maker signal during the cardiac cycle, suggesting poor perfusion to a muscle of the heart and the location of this myocardial infarction.

Technology developed in the 70's called Superconducting Quantum Interference Device (SQUID) technology was the first device developed to capture the weak electromagnetic signals produced from this electrical activity. But these machines are very large, require use in a shielded environment to eliminate background electromagnetic noise, and requires cryogenics to cool these machines to achieve the detection sensitivities required. The current price point of traditional SQUID machines is in the $1M range and costs ˜$5K per use to operate due to the cost of helium to cool the device. (See, for example, https://www.elekta.com/diagnostic-solutions/elekta-neuromag-triux/). Resultantly, there are only 20 of these units currently in operation in the U.S.

Other technologies include optical magnetometers (see, for example, https://quspin.com/) which do not require cryogenic cooling, but still require use in a shielded environment and cost $7,500 per unit. A 32-array device made from QuSpin devices cost in the $240K range.

TDK, the computer hard disk storage vendor has developed technology referred to as magnetoresistive technology. The technology has sensitivity near the range of SQUID technology but does require shielding but not cooling. TDK employs a spintronics-type MR element which has a sandwich structure made of a thin-film of non-magnetic material sandwiched between thin ferromagnetic films. One of the ferromagnetic films is a pin layer (fixed layer) in which the direction of magnetization is fixed by pinning, and the other ferromagnetic film is a free layer in which the direction of magnetization follows the direction of the external magnetic field. Since the electrical resistance of the element varies proportionately with the relative angle between the direction of magnetization of the pin layer and free layer, the intensity of the magnetic field can be measured from the magnitude of the current.

Creavo Medical Tech markets a device utilizing an induction coil array for cardiac monitoring. The device uses a cardiac electrophysiology “gating” signal to identify the wavelet portion of the signal coincident with a cardiac pace-maker signal. This can reduce the amount of competing background noise and eliminate the need for use in a shield room. The Creavo device is limited to capturing the cardiac electrophysiology signal, acting as a magnetocardiogram (MCG) device.

### BRIEF DESCRIPTION OF THE DRAWINGS

FIG. 1 shows an overview of the coil array placement upon a human subject. This example configuration shows a coil array that fits up to 7 groups of coils (namely ‘big fixture holder’). Inside each holder, a ‘small coil fixture’ can be inserted that includes multiple coils (up to 7 in this example) for signal averaging. The design is however adaptable, i.e., different numbers of coils and in different configurations may be employed per application scenario (intended imaging resolution, time available, noise levels, and so on).

FIG. 2 shows the block diagram of the coil sensing system. The example is with reference to bio-magnetic signal detection from the heart. The coil array picks up bio-magnetic fields and sends them to an Analog-to-Digital Converter (ADC). A standard electrocardiography sensor is also picking up signals and sending them to the same ADC for further post-processing and denoising.

FIG. 3 shows a parameterized version of one of the coils as well as optimized design parameters for an example scenario of magnetocardiography sensing.

FIG. 4 shows (a) an example coil sensor, (b) a circuit board with 20 integrated amplifier board, and (c) an overview of the amplifier circuit board.

FIG. 5 plots the sensitivity of the coil sensor and amplifier system as measured in a non-shielded environment.

FIG. 6 provides a block diagram of the employed experimental set-up.

FIG. 7 shows: (a) an 8-shaped coil used to emulate the heart's electromagnetic activity, (b) an overview of the experimental set-up, and (c) an overview a 4-coil sensing system placed upon the emulated heart.

FIG. 8 plots: (a) the raw signals acquired from each individual coil, (b) the processed data after averaging all viewing windows, (c) the processed data after averaging from multiple sensors, and (d) the final processed data.

FIG. 9 shows the: (a) maximum Z-component of the magnetic field at planes with different distance away from the emulated heart, and (b) the Z-component of the magnetic field generated by the emulated heart at the plane where the recording coils are placed (z=5 cm).

FIG. 10 plots the processed final averaged signal of all 4 coil sensors using (a) 30 min, (b) 15 min, and (c) 6 min of recording time, as well as the processed final averaged signal using 15 minutes recording time when (d) 1 coil sensor is used to detect 1 area, (e) 2 coil sensors are used to detect 2 areas, and (f) 4 coil sensors are used to detect 2 areas.

### DETAILED DESCRIPTION

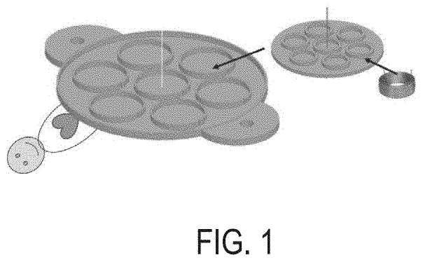

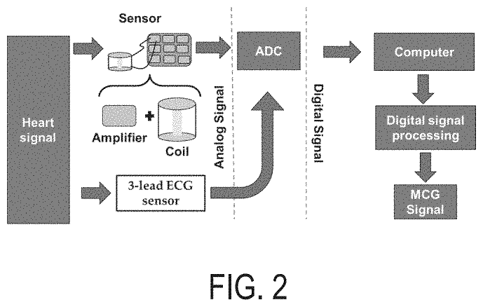

The following disclosure describes the present invention according to several embodiments directed to arrays of inductive coil sensors and related processing and control hardware for detecting electrical activity in a patient non-invasively. Some embodiments utilize air core coils having dimensions shown in FIGS. 1 and 2, or within a similar range. A ratio of inner to outer coil diameters Di/D around 0.56 has been found to minimize noise. Some embodiments utilize coils having this Di/D ratio or within the range of 0.5-0.6. Furthermore, noise has been found to be reduced when the Ratio l/D=0.7182. Accordingly, some embodiments utilize coils having this ratio or within the range of 0.7-0.74. Larger coil outer diameter leads to higher coil sensitivity. Both of the coil designs of FIG. 2 have been tested to successfully detect biological magnetic field. In some embodiments, depending on specific need, coils in the range of 0.3<Di/D<0.8, keep 0.679<l/D<0.787 can be used. In some embodiments, Ferromagnetic material with high permeability, such as metglas, iron etc. can be added to the air core design. By adding the core, sensor sensitivity can increase, but the sensor's weight will generally increase.

Additional information about how the ideal ratios can be determined is found in Appendix A. To maximize sensitivity (S) of a coil sensor along the z direction, when given a fixed magnetic flux density (B) at a fixed frequency (f), S can be calculated as follows. Here, high sensitivity implies large output voltage with respect to noise (high signal to noise). The output voltage of an induction coil (V) can be calculated as

$$V = An \frac{Δ B}{Δ t} = 2 {π}^{2} {fn R}_{a}^{2} B$$

where Ra1 is the average radius of the coil, A is the surface area of the coil, and n is the number of coil turns. Considering the coil to be tightly winded, this equation can be rewritten using the four optimization parameters as:

$$V = \frac{{π}^{2} DB}{16 {d}^{2}} ( D - Di ) {( D + Di )}^{2}$$

The noise, namely thermal Johnson noise (VT), produced by our coil can be expressed as:

$${V}_{T} = 2 \sqrt{{k}_{B} T Δ f R} = \frac{2 \sqrt{{k}_{B} T Δ fl ρ ( D + Di ) ( D - Dt )}}{{d}^{2}}$$

where R is the coil resistance, kB is the Boltzmann constant, T is the coil's absolute temperature in Kevin, dis the diameter of the wire, and p is the wire resistivity.

For maximum sensitivity along the z direction, the coil parameters should follow:

$$\frac{1}{D} = \frac{3}{\sqrt{20}} \sqrt{\frac{1 - {( \frac{Di}{D} )}^{S}}{1 - {( \frac{Di}{D} )}^{S}}}$$

Thus, the sensitivity can be expressed as:

$$S = \frac{V}{{BV}_{T}} = \frac{\sqrt{3} {π}^{2} f ( D + Di ) \sqrt{D}}{32 \cdot {20}^{\frac{1}{4}} \sqrt{{k}_{B} Δ f ρ T}} \cdot {[ \frac{1 - {( \frac{Di}{D} )}^{3}}{1 - {( \frac{Di}{D} )}^{3}} ]}^{\frac{1}{4}} \sqrt{( {D}^{2} - {Di}^{2} )}$$

Here, it is worth noting that d does not play any role in determining the coil sensitivity (S). Given fixed values for the coil length/height (l), outer diameter (D) and inner diameter (Di), an increase in the wire diameter will decrease the signal level. In the meantime, with fixed l, D and Di, an increase in wire diameter will also decrease the thermal Johnson noise. When calculating the sensitivity, these two effects will eventually cancel out the impact of d. Assuming a fixed frequency and temperature, this equation can be rewritten as:

$$\frac{S}{{D}^{2.5}} = {M ( 1 + \frac{Di}{D} ) [ \frac{1 - {( \frac{Di}{D} )}^{3}}{1 - {( \frac{Di}{D} )}^{3}} ]}^{\frac{1}{4}} \sqrt{( 1 - {( \frac{Di}{D} )}^{2} )}$$

where M is a positive real value. Eventually, this can be used to identify the optimal coil design with the highest sensitivity given fixed values for B and f. The optimal design is found when Di/D and l/D equals to the aforementioned ratio. Note here, the optimal ratio remains the same despite the coil size. Larger size coil will have higher sensitivity while keeping the ratios the same. Prior art has used a different range than these embodiments and have used a ferromagnetic core. These coil designs use an air core coil, which can reduce weight and cost.

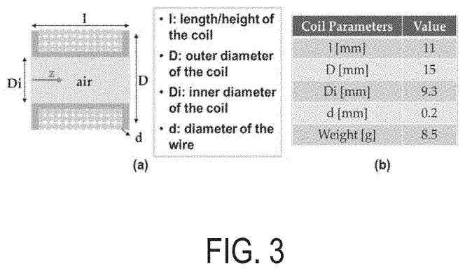

Coils can be placed in an array. Different numbers of coil sensors can be used for neuron magnetic signal detection. In various embodiments, different coil placement techniques with corresponding signal processing method can be adapted for different clinical requirement needs. As one example, FIG. 3 shows one of the possible coil placement techniques with a designed fixture for holding the coil sensors. FIG. 3(b) shows how the fixture can be placed upon a human body. In this embodiment, the overall fixture consists of 7 small coil fixtures, which each hold 7 induction coil sensor and one big fixture holder to hold the 7 small coil fixtures. The developed fixture has the ability to detect up to 49 localized points. While other geometric arrangements of coils are possible, the 7×7 arrangement of FIG. 3 is illustrative of an embodiment that allows configurability. In some embodiments, a combination of large coils and smaller coils can be used, placing 1-7 coils in each of the 7 coil fixtures. In some embodiments individual coils can be selected by software control for a given application.

To help identify each coil, small coil fixtures are referred to as slot x (x is a letter from a, b g), which is then placed in big fixtures referred to as slot y (y is a number from 1, 2 7), such that each small coil can be referred to as coil yx. In a first example, all 49 coils are used, placed inside all 7 small coil fixtures from all 7 slots in the big fixture holder. The final signal can be interpreted as 49 localized signals coming from each one of the coils, or 7 localized signals as each small coil fixture producing one averaged signal. The final signal can also be interpreted as one big signal (neuron ave center) coming from the center of the targeted neuron activity site subtracting the outside environmental noise (noise ave). In some embodiments, neuron ave center signal can be obtained by averaging the data collected from coil 7a, coil 7b coil 7g, together with coil 1d, coil 2e, coil 3f, coil 4a, coil 5b, coil 6c. The rest of the coils can be used to produce the averaged noise (noise ave).

In a second example, only 7 coils are used in slot i, where i is a real number from 1 to 7 filling the one small coil fixture slot a, b . . . g. The final signal can be interpreted as 7 individual neuron signals coming out from coil ia, coil ib coil ig or from individual neuron site signals (site1, site2 and site3) with site1 being the averaged data from coil ia and coil ib, site2 being the averaged data from coil is and coil id, site3 being the averaged data from coil ie and coil if. In a third example, four coils, namely coil 2g, coil 2e, coil 5g and coil 5b are used to produce the final signal. For the two coils in slot 2, coil 2g and coil 2e, a positive averaged neuron signal (neuron_ave_positive) is produced. For the two coils in slot 5, coil 5g and coil 5b, a negative averaged neuron signal (neuron_ave_negative) is produced. The final results can be interpreted as one final averaged signal using neuron_ave_positive subtracting neuron_ave_negative. Other fixtures/array configurations can also be used in other embodiments.

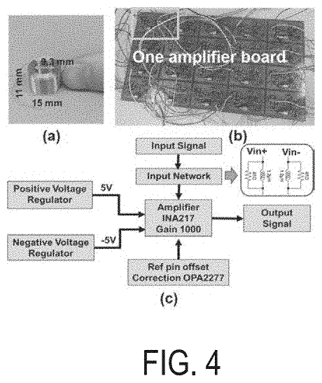

Exemplary options of signal processing and noise reduction are shown in FIG. 4. Options used in various embodiments include the following. A bandpass filter allows the system to focus the raw data in a targeted frequency range. A notch filter can remove specific frequency background noise, such as transmission line noise and its harmony (in US 60 Hz, 120 Hz, 180 Hz). An empirical mode decomposition (EMD) filter can further reduce noise and smooth the final signal. Signals can be averaged over multiple coils based on different/specific array configurations. Different filters can be used alone and in combination. All of these filters can be chosen according to specific needs. For example, if only bandpass filter is sufficient to remove noise, only apply one of the filters to the digital signal processing is enough; if target signal is at extremely low field level or recording is happening in an extremely noisy environment more filters might be used for additional noise reduction, for example. Bandpass, notch, averaging and EMD filter can all be integrated together in the combinations as shown in FIG. 4.

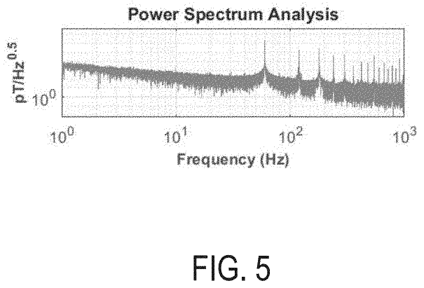

Multiple filters can be combined together to filter out noise and depict a clear signal when recording extremely low field level bio signal in an unshielded environment. For example, if an EMD filter is placed right after the raw signal, followed by bandpass and notch filters, this EMD filter is of minimal use. To maximize each filter's efficiency in de-noising the raw data, filter arrangement can play an important role. An EMD filter performs the best when placed at the end of the signal processing procedure. One example of a suitable filter arrangement is shown in FIG. 5, where the bandpass filter feeds to notch filters, which feed to a filter that averages each viewing window, before sending the signal data through an EMD filter.

Different types of signals can be used as a gating signal for the signal processing. The gating signal can be used to identify wavelets where the desired signal is located in the stream of data captured by the inductive array. In some embodiments, the gating signal is an electrical signal, such as electrical signal naturally emanated by neuron activity. In some embodiments, the gating signal can be a stimulus input signal, such as in ocular stimulation signal. Exemplary signals that can be used as a gating signal include: an electrocardiography (ECG) and pulse can be used as a magnetocardiograph (MCG) gating signal; Eye movement (i.e. blinking of the eye) can be used as a magnetoencephalography (MEG) gating signal; ocular stimulation signal can be used for magnetomyography (MMG), magnetospinography (MSG) and other nerve conduction study gating signals.

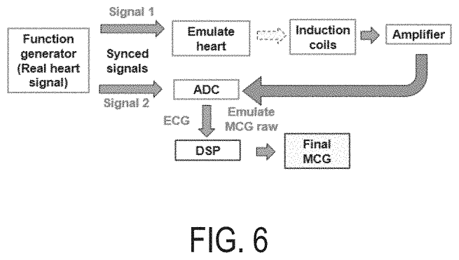

In some embodiments, an amplifier board is specifically separated from the receiver coils to reduce EM affects and reduce noise, which can degrade sensitivity. In some embodiments, an input network is used to reduce input oscillations caused by low source impedance. An input network can include two inductor/resistor pairs connected to the positive and negative ports of the input signal and is further connected to an amplifier (e.g., INA217). Coil sensors generally have very low impedance (e.g., ˜3.5 9), which is desirable for the chosen amplifier that has a very low voltage noise and a relatively high current noise. Given the low source impedance, the current noise will not contribute much to the application. On the other hand, a very low source impedance (<1OQ) can cause the instrumental amplifier to oscillate; our input network greatly reduces any oscillation tendencies. In some embodiments, to further eliminate vibration noise across multiple amplifiers, all amplifiers are integrated into a single board (20 amplifiers shown in the example system of FIG. 6.) The system of FIG. 6 represents the amplifying circuit for a single sensor coil. The circuit can be replicated on a single amplifier board (or on separate boards) for each receiver coil in the array.

Environmental noise reduction techniques can improve signal-to-noise ratios when detecting the extremely low biological magnetic fields. In addition to the circuit and signal processing techniques disclosed herein, can be advisable to limit exterior environmental noise by recording in a magnetic field shielding room, partially shielding the target recording site, or using a simple audiology booth.

Embodiments of a neural magnetometer utilize an induction coil array placed on top of, and as close as possible to the region of interest to capture neurotransmissions. A gating signal is used to identify those wavelets where the desired signal is located in the stream of data captured by the array. An exemplary gating signal can be an electrocardiogram (ECG) that identified the start and stop of a cardiac cycle, which is the portion of the signal or wavelet of interest when using the device as a magnetocardiogram. If the device is used to capture a neurotransmission, an ocular stimulation signal can be used as the gating signal.

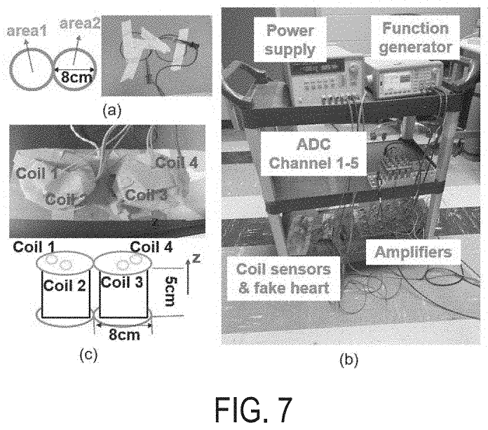

FIG. 7 shows an example of how an embodiment of a magnetometer can capture a magnetocardiograph (MCG). An exemplary sensor operates based on Faraday's law: the changing magnetic flux of the heart is captured by an array of induction coils placed against the chest, leading, in turn, to the generation of time-varying voltages across the coils' terminals. Capturing this changing voltage is a means of monitoring the magnetic field activity of the heart. Theoretically, one sensing coil would be sufficient to couple to the magnetic field of the heart. But, in practice, the low amplitude of the heart's magnetic field necessitates a multitude of sensing coils to concurrently capture the signal so that noise can be brought down via DSP within a reasonable amount of recording time (i.e., a few minutes). A tradeoff in this case is that the number of coils used to average the recorded signals limits the sensor's imaging resolution.

As shown in the system of FIG. 7, the heart's magnetic field is first picked up by the array of MCG coil sensors. Due to the signal's extremely weak magnitude (i.e. ˜10-6 Gauss), MCG coils are connected to an amplifier board that amplifies their recorded signal by 1000 times. The amplified signals are then picked up by a multi-channel analog to digital convertor (ADC) and sent on for further processing. The ultimate aim from this stage on is to retrieve the MCG signal from the noise floor via advanced Digital Signal Processing (DSP). This DSP process utilizes the collection of a concurrent gating signal. The ECG is synced with MCG (via a derivative relationship) and the ECG serves to identify the MCG cycles which are otherwise hidden under the noise floor. A 3-lead ECG sensor is indicated in FIG. 7, but a pulse (or other) sensor may be alternatively used.

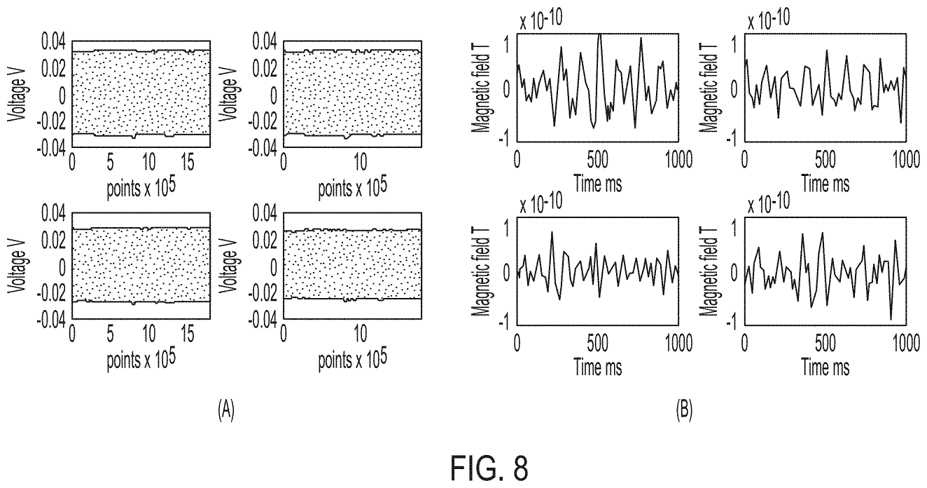

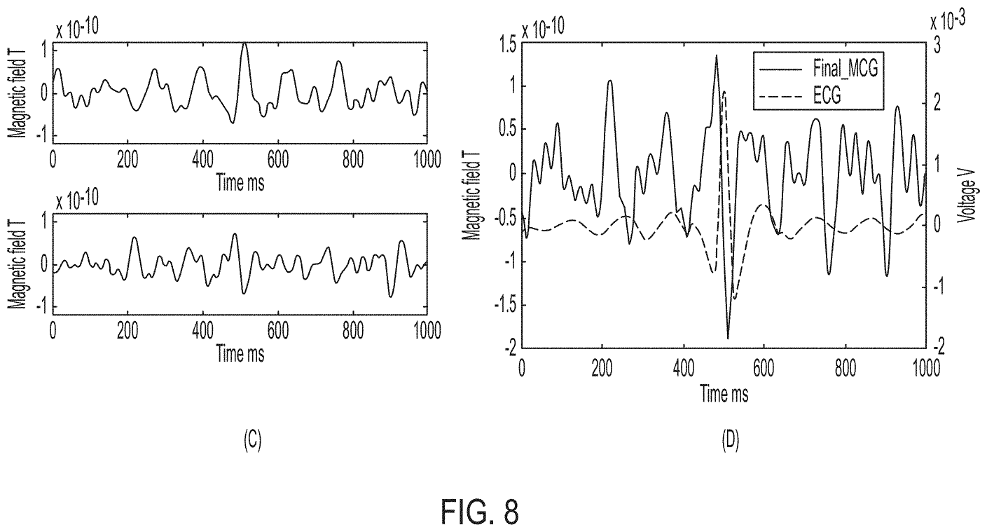

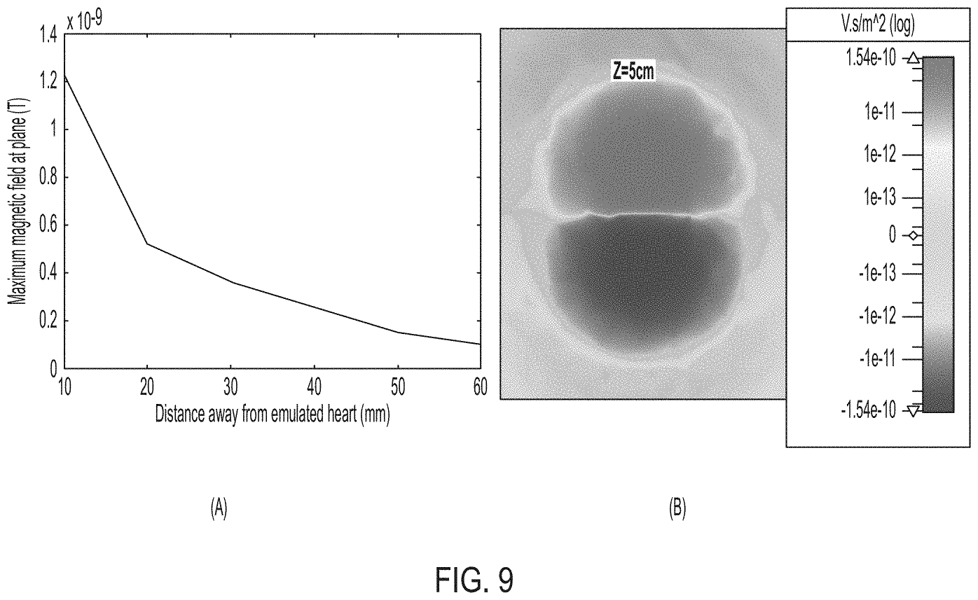

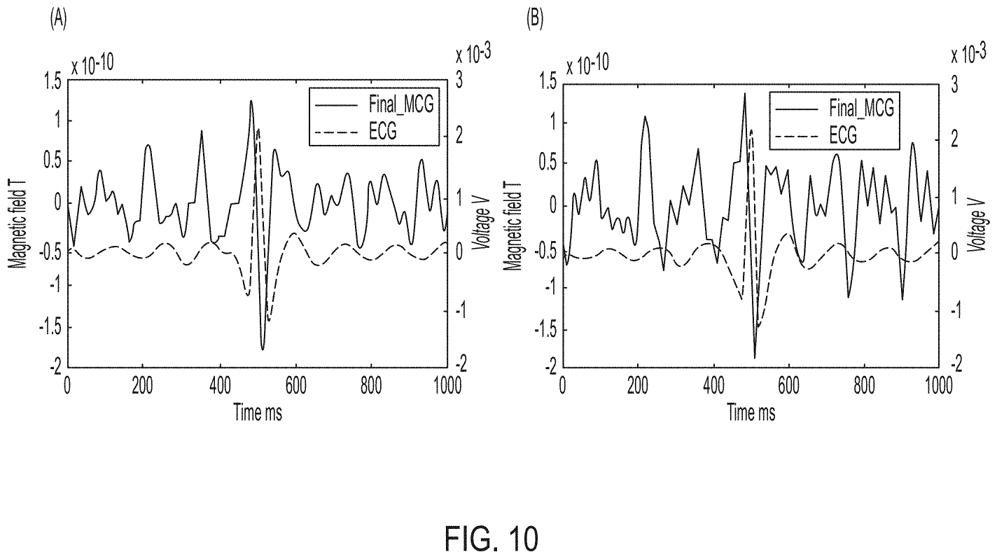

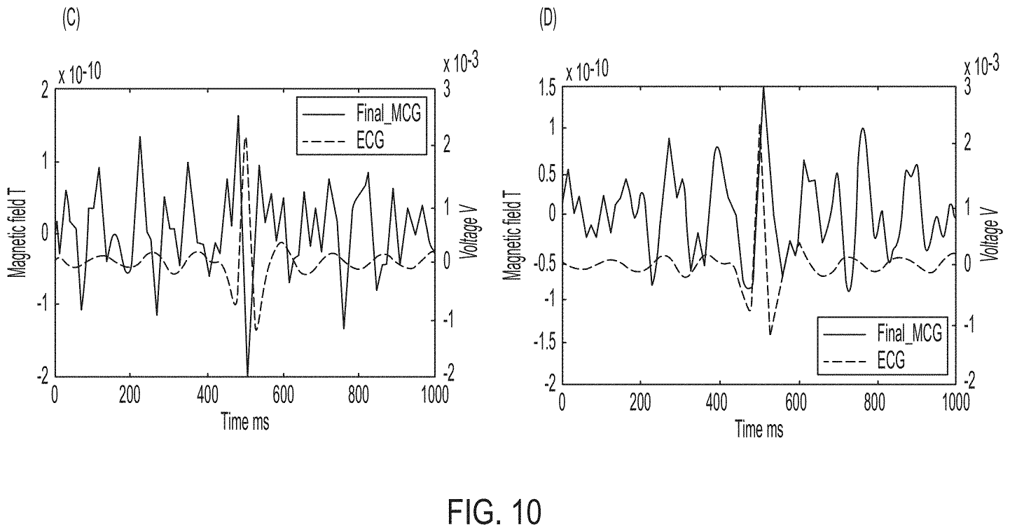

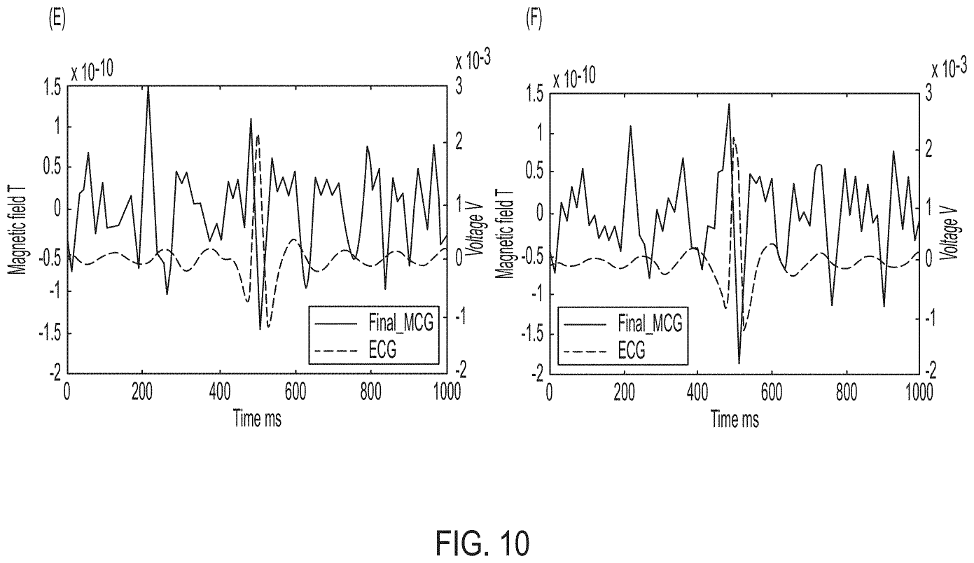

FIG. 8 shows another example of how this sensor works to capture evoked nerve magnetic field. This block diagram shows how the system can sense an evoked nerve potential magnetic wave. The target nerve is activated using an external stimulating signal. This stimulating signal can be either an electric pulse or a magnetic wave. This signal can be applied simply using an electrode or a magnetic stimulation coil. The activation produces ironic current passing through the nerve, which in turn produces magnetic waves propagating to the outside of the nerve/cell. Our coil sensor(s) pick up this alternating magnetic field and generate changing voltage which can later be converted to magnetic field strength. The amplifier is used to amplify the extremely low evoked nerve potential so that it can be captured using the Analog to Digital Converter (ADC). Simultaneously, the ADC records the stimulation signal, which can be used later as the gating signal during DSP. Through advanced DSP, the final evoked nerve magnetic wave can be obtained.

An exemplary application of the magnetometer includes simple neuro health testing. This can include manual or processor-controlled activation of the sensory nervous system (e.g. ocular stimulus using light, pin pick to fingers or toes, auditory stimulus). Nerve conduction studies are currently performed in the hospital setting using an electrode-based approach to ensure surgical procedures and/or patient placement that may reduce perfusion does not damage the nervous system. However, electrode-based systems may inadvertently activate a nearby muscle and provide a false positive response. In some embodiments, a second electromagnetic antenna (the magnetometer array discussed throughout being the first) stimulates the nervous system to enable use of the EM detection system to assess or diagnose the response. This may be performed during spinal or joint replacement procedures, for example. Similarly, this system can be used to identify dysfunction within the neuro pathway of an individual with a radiculopathy (spinal damage) or neuropathy, such as the inability to feel a pin prick made on a specific toe. The ability to employ EM to locate the cause of the dysfunction such as a lesion, stenosis, or vertebrae compression has the potential to overcome limitations of current imaging or electrode-based nerve stimulation diagnosis that are unable to identify the source of pain or neural dysfunction. Spinal Cord Injury without Radiographic Abnormality (SCIWORA) and Failed Back Surgery Syndrome (FBSS) are two instances where current imaging or nerve stimulation diagnostics have demonstrated the ability to located the source of nerve pain or injury.

Embodiments that combine a magnetometer system with EM nerve stimulation antenna can be used to provide feedback for adequacy of signal to block a neuro transmission in order to provide intervention guidance for pain block applications.

Embodiments can also be integrated into a wearable device. A wearable device can include an antenna used to collect the EM signal made of conductive thread and incorporated into a textile design. Such a device may be used to capture abnormal cardiac electrophysiology. (EEG devices should be removed after 14 days of use to prevent potential skin irritation.)

Embodiments can also be integrated into a hospital bed. The device can be placed in the mattress of a hospital bed to provide non-invasively continuous cardiac electrophysiology signals for a heart failure patient. Non-contact avoids possible skin irritation from electrodes. Furthermore, EM cardiac pace-maker signals captured can enable recognition of early signs of myocardial (cardiac muscle) dysfunction and/or poor or irregular pace-maker.

Embodiments can also be utilized as an intervention guidance device for nerve regeneration therapy. Use of transcranial electromagnetic therapy is an approved reimbursed procedure that is used to treat depression. Electromagnetic therapy could be used to treat or repair nerve tissue damage in non-cranial locations. The ability to obtain feedback using the antenna can provide intervention guidance to enable adjustment of the parameters of the treatment device.

The system of the figures is not exclusive. Other systems may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. No claim element herein is to be construed under the provisions of 35 U.S.C. 112, sixth paragraph, unless the element is expressly recited using the phrase “means for.”

1. A method of monitoring neurotransmissions, the method comprising:
a. arrays of miniaturized coils placed upon the body part of interest;
b. amplifiers placed at a certain distance away from the coil array to minimize noise;
c. a digital signal processing method that filters and averages the raw signals to denoise them.

2. The method of claim 1, comprising of an array of air core induction coils, whose ideal coil inner to outer diameter ratio is 0.62.

3. The method of claim 1, comprising of an array of magnetic core induction coils, whose ideal coil length to coil diameter ratio is 0.73.

4. The method of claim 1, comprising of signal processing capability that incorporates bandpass filter to enhance detection sensitivity. Prior to any signal post-processing, bandpass filtering is performed within the anticipated range of frequencies for the target signal to eliminate noise. Following bandpass filtering, the signal can be averaged based on viewing windows determined by a trigger/sync signal.

5. The method of claim 1, comprising of an induction coil array that is further comprised of multiple subarrays, for example placed in a flower shape configuration. This design is versatile such that some or all of the subarrays may be filled partially or fully with coils. The approach provides flexibility in terms of the number and location of the coils placed upon the array to accommodate different scenarios (e.g., clinical application, body part, noise environment, target resolution, processing time).

6. The method of claim 1, where the post-processing algorithm is adaptable to accommodate the diverse coil configurations of coil 5.

7. The method of claim 1, where the associated amplifiers, CPU, power supply, etc. are placed a minimal distance away from the coils (at least 1 foot) to minimize injection of noise into the detection circuit.

8. The method of claim 1, where the coils are stabilized as close as possible to the human body by means of a flat fixture, such that they remain stable regardless of natural motion of the human body (e.g., breathing).

9. The method of claim 1, where the amplifiers for all coils are printed on the same board, placed away from the coils per claim 7 and are also adaptable in their use per claim 5.
